(19) United States
(12) Patent Application Publication
(10) Pub. No.: US 2018/0042755 A1
(43) Pub. Date: Feb. 15, 2018

(54) BACK BRACE

(71) Applicant: BUCKNELL UNIVERSITY, Lewisburg, PA (US)

(72) Inventors: CHARLES JOHN KIM, LEWISBURG, PA (US); JB RING, BALTIMORE, MD (US); JOEP NIJSSEN, DELFT (NL); SAMUEL PRATT, HUDSON, OH (US); SUNIL AGRAWAL, NEWARK, DE (US); PAUL STEGALL, KANSAS CITY, MO (US); JOONHYUK PARK, NEW YORK, NY (US)

(21) Appl. No.: 15/677,869

(22) Filed: Aug. 15, 2017

Related U.S. Application Data
(60) Provisional application No. 62/375,254, filed on Aug. 15, 2016.

(51) Int. Cl. A61F 5/02; A61F 5/01

(52) U.S. Cl. CPC A61F 5/028; A61F 5/0104

(57) ABSTRACT

A back brace comprising: a pelvic member configured to be secured to a user such that said pelvic member is essentially immobilized relative to the pelvis of said user; a thoracic member configured to around the user such that said thoracic member is essentially immobilized relative to the thorax of said user; and one or more compliant connectors between said pelvic member and said thoracic member and configured to provide a resistive force between said pelvic member and said at least one thoracic member.

### REFERENCE TO RELATED APPLICATIONS

This application is based on U.S. Provisional Application No. 62/375,254, filed Aug. 15, 2016, and incorporated by referenced herein along with its appendices.

### REFERENCE TO APPENDICES

Appendix A, A Passive Brace To Improve Activities Of Daily Living Utilizing Compliant Parallel Mechanisms, and Appendix B, Preliminary Summary Compliant Scoliosis Brace, are hereby incorporated by reference in this application.

### BACKGROUND

Scoliosis is a musculo-skeletal disease that causes a three-dimensional deformity primarily characterized by the curvature of the spine in the frontal plane. In this paper we will focus on adolescent idiopathic scoliosis (AIS), which is the most common form of scoliosis and affects 2 to 3% of adolescents, approximately 10% of which will require medical treatment. Girls are nearly 3 times more likely to have scoliosis than boys. Scoliosis usually affects adolescents during their growth periods between the age of five and eight, and ten until the end of growth.

Scoliosis can lead to certain health implications. Most notably, patients with scoliosis that requires bracing or surgery can experience shortness of breath. In addition, scoliosis patients can suffer from heart problems and back pain. Nonphysical health implications include struggling with self-image, an emotional pain.

A scoliotic spine contains either an “S” or “C” curve. The degree of scoliosis is generally characterized by the Cobb angle, defined as the angle between the two most tilted vertebrae of a spine segment. Cobb angles less than 25° require biannual checkups but no treatment unless the angle increases. A Cobb angle greater than 40° requires surgery. Cobb angles between 25° and 40° generally require bracing to prevent further progression.

Bracing is the most common treatment for AIS. The goal of bracing is not necessarily to correct the curve, but to prevent further progression, though correction can occur. Braces are generally to be worn up to 23 hours a day. It has been recognized that a brace should be designed with regard to the “three C's” (Comfort, Control, and Cosmetics). Comfort refers to the patient's ability to perform ADL, control refers to the brace's ability to apply correction forces of the right directions and magnitudes, and cosmetics refers to the appearance of the brace itself, along with how the patient perceives themselves in the brace. While conventional braces have been able to achieve one or even two of the three Cs, none have been able to achieve all three in the same brace.

The most common braces are rigid, although concepts of flexible braces have recently been explored, and a few have been brought to market. Rigid braces include the Milwaukee, Boston, and Cheneau braces. Flexible braces include the SpineCor and the TriaC braces. The rigid braces tend to be more effective and achieve the control goal of the three Cs, while the flexible braces tend to achieve comfort and cosmetic goals at the expense of control.

For example, the Milwaukee brace was the first documented brace to prove effective with a 74% success rate. It consists of a steel and leather pelvic base with rods that extend to the throat. However, the ‘superstructure’ of this brace caused lower jaw and dental deformities. The Boston brace, currently most recommended for treatment, consists of a standardized size polystyrene shell, tightened around the torso using straps, with interior foam padding to apply corrective forces and ‘cut-outs’ to provide relief. The Boston brace has up to a 93% success rate. The Cheneau brace, which has many variations, is also a rigid plastic shell, but is customized to each individual patient

The most common cause of failure in rigid brace treatment is a lack of patient compliance in wearing the brace. Comfort and aesthetics are the main reasons that patient do not wear braces for the prescribed amount of time each day. Rigid braces limit range of motion, including flexion and rotation, which in turn limits the patient's ADL. Braces are also bulky and cannot be easily hidden. Oversized clothes must be worn to hide a brace. This can damage self-confidence and have a psychological impact on the young patients who are usually going through puberty at the time of treatment.

Flexible braces are designed to address the drawbacks of rigid braces. The SpineCore brace is a flexible brace consisting of elastic bands, a pelvic base, and crotch and thigh bands. This brace allows the patient a much greater range of motion compared to rigid braces, but has a lower success rate according to some sources. Guo, et al found in a study that 5 out of 7 patients who encountered progression of the spinal deformation with the SpineCor brace had no further progression after switching to a rigid brace. The TriaC brace is another flexible brace that consists of two parts—lumbar and thoracic straps that are interconnected by a flexible coupling device. Although the manufacturers of the TriaC brace purport a success rate of 76% success rate, such flexible braces are nevertheless less effective than rigid braces in preventing scoliotic progression.

What is needed is a brace that achieves the three Cs—Comfort, Control, and Cosmetics. The present invention fulfills this need among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

The present invention relates to a flexible scoliosis brace designed to provide corrective force in a specific directions and mobility in other directions. The invention also relates to the identification of the problem with the SpineCor and TriaC braces. Specifically, without being tied to a particular theory, Applicants believe these braces lack the ability to be tuned for both compliance and stiffness that compliant mechanisms, described below, can supply. Both the SpineCor and TriaC braces are designed to provide corrective force with varying levels of reported success. This is in contrast to rigid braces which are designed to constrain the spine. This relationship is characterized as force-controlled correction (flexible braces) vs. displacement-controlled correction (rigid braces). The invention uses compliant mechanisms to constrain the spine through tuned stiffness, while permitting specific motions through kinematic design. Compliant mechanisms are used because they can apply the corrective force, but also allow the patients some range of motion. Thus, we seek to improve patients' comfort by designing a brace that improves range of motion, while remaining stiff in the corrective direction.

The brace comprises compliant mechanisms, which may or may not be attached to rigid elements. The brace may also include flexible shell elements, flexures, and/or lamina emergent sheets. In one embodiment, the back brace comprises: (a) a pelvic member configured to snuggly wrap around a user such that the pelvic member is essentially immobilized relative to the pelvis of the user; (b) at least one thoracic member configured to snuggly wrap around the user such that the at least one thoracic member is essentially immobilized relative to the ribs of the user; and (c) one or more first compliant connectors between the pelvic member and the at least one thoracic member and configured to impart an urging force between the pelvic member and the at least one thoracic member.

In another embodiment, the back brace comprises: (a) a pelvic member configured to snuggly wrap around a user such that the pelvic member is essentially immobilized relative to the pelvis of the user; (b) at least one thoracic member configured to snuggly around the user such that the at least one thoracic member is essentially immobilized relative to the ribs of the user; and (c) least one compliant connector between the pelvic member and the at least one thoracic member, and being configured to allow the pelvic member and the at least one thoracic member to move relative to each other with at least 2 degrees of freedom, but less than 6 degrees of freedom.

### BRIEF DESCRIPTION OF FIGURES

FIG. 1 shows one embodiment of the brace of the present invention.

FIG. 2(a) shows a brace which has a helical pelvic member which wraps entirely around the user.

FIG. 2(b) shows a brace with another embodiment in which the pelvic member does not wrap around the patient, but rather is configured as a pad to apply pressure to a particular point on the user's hip/pelvis.

FIG. 2(c) shows a brace that has a circular pelvic member which wraps around the user.

FIG. 2(d) shows a brace that has a pelvic member that wraps around the user's hip but is open in the front.

FIG. 2 (e) shows a brace where the pelvic member is configured to wrap around most of the body, but remains open in the front as shown.

FIG. 3(a) shows the brace on the torso from the right side view.

FIG. 3(b) shows the brace on the torso from the front.

FIG. 3(c) shows the brace from the left side without being superimposed on the body.

FIG. 3(d) shows the brace from the rear without being superimposed on the torso.

FIG. 4(a) shows the brace which comprises a pelvic member and a thoracic member, and intermediate members.

FIG. 4 (b) shows the brace as having essentially the same pelvic thoracic and intermediate members, but having different compliant connectors.

FIG. 4 (c) shows the brace which comprises a pelvic member and thoracic member and an intermediate member.

FIG. 5(a) shows that the brace comprises a pelvic member and a thoracic member with a combination of flexure and shell compliant members connecting the two together.

FIG. 5(b) shows the brace in which the pelvic member is connected to a pair of cartwheel hinges, which, in turn, is connected to a crossed helix.

### DETAILED DESCRIPTION

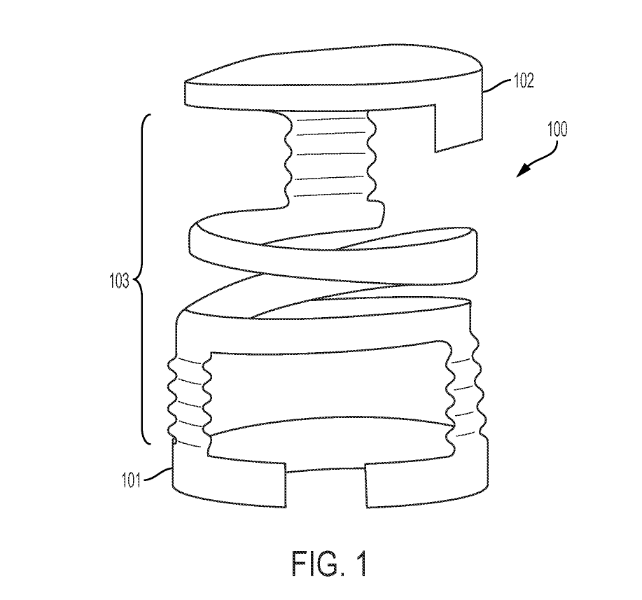

Referring to FIG. 1, one embodiment of the brace 100 of the present invention is shown. The brace 100 comprises a pelvic member 101 configured to be secured to a user's pelvis such that the pelvic member is essentially immobilized relative to the pelvis of the user, and a thoracic member 102 configured to be secured to a user's chest such that the thoracic member is essentially immobilized relative to the chest of the user. The brace 100 also comprises at least one compliant connector between the pelvic member and the thoracic member 102 to provide an urging force between the pelvic member and the thoracic member.

Each of these elements is described below in more detail and in connection with selected alternate embodiments.

The pelvis and thoracic members 101, 102 function to secure the brace to the user's pelvis (hip) and thorax (chest), respectively, and to transmit the force applied between them by the compliant\ connector(s) to the user's body at the pelvis and thorax. To this end, the members generally, although not necessarily, comprise a rigid or semi-rigid material to resist deformation from the force of the compliant connector. The type of material used and its thickness will depend on the expected forces and the physical configuration of the pelvic and thoracic members, which can vary as described below. One skilled in the art will readily understand how optimize the materials and their thickness to ensure the pelvic and thoracic members have the requisite stiffness to absorb the stresses imposed by the compliant connectors and translate those forces to the user's body. Suitable material include, for example, carbon fiber composite, fiberglass composite, and plastics such as Acrylonitrile butadiene styrene (ABS), acetal, polycarbonate (PC), and polypropylene (PP).

The pelvic and thoracic members may also comprise belts or additional apparatus to make the brace's attachment to the body more secure. Such apparatus is well known to those of skill in the art, and, thus, is not described herein in detail.

The compliant connectors 103 serve to connect the pelvis and thoracic members and provide a resilient force among the components. The force generation approaches of the compliance connectors are described in detail in Appendix B, Chapter 4. Generally, the compliance connectors are configured to provide one or more of force mechanisms selected from shell mechanisms, such as cross helix, helical strip, single curve, hyperbolic paraboloid, double paraboloid, single corrugated and double corrugated, or flexure mechanisms, such as cartwheel hinge, parallel beam, cross pivot hinge, cross beam, LET outside, LET inside, or S-beam. In one embodiment, the compliance connectors are configured to generate at least 30N, 40N or 50N of force between the pelvic and thoracic members.

The compliance connectors may be configured to achieve the desired stiffness between the pelvic and thoracic members while still allowing for primary motions. Generally, the primary motions involve sagittal bending, twisting, and lateral bending. Modeling the brace to balance desired stiffness while maintaining primary motions is described, for example, in Appendix B, generally, and Chapters 3, 5, 6, 8, and 9 in particular. In one embodiment, the compliance connectors are configured to allow for at least 13° in the sagittal direction, 10° in twist, and 9° in lateral bending. In one embodiment, the compliant connectors are configured to allow the pelvic member and the at least one thoracic member to move relative to each other with at least 2 degrees of freedom, but less than 6 degrees of freedom. In another embodiment, the compliant connectors are configured to allow the pelvic member and the thoracic member to move relative to each other with at least 2 degrees of freedom, but less than 5 degrees of freedom. In still another embodiment, the compliant connectors are configured to allow the pelvic member and the thoracic member to move relative to each other with at least 2 degrees of freedom, but less than 4 degrees of freedom.

As with the pelvic and thoracic members, the materials used for the compliance connectors will depend on the desired forces and brace configuration. For example, in some embodiments, the compliance connectors comprise the same material as the pelvic and thoracic members. In such embodiments, the compliance connectors may be integral with the pelvic and thoracic members. In other embodiments, the compliance connectors are discrete and comprise elastic materials such as ABS, PP, PC, or acetal and stiffer materials such as titanium, stainless steel, and aluminum.

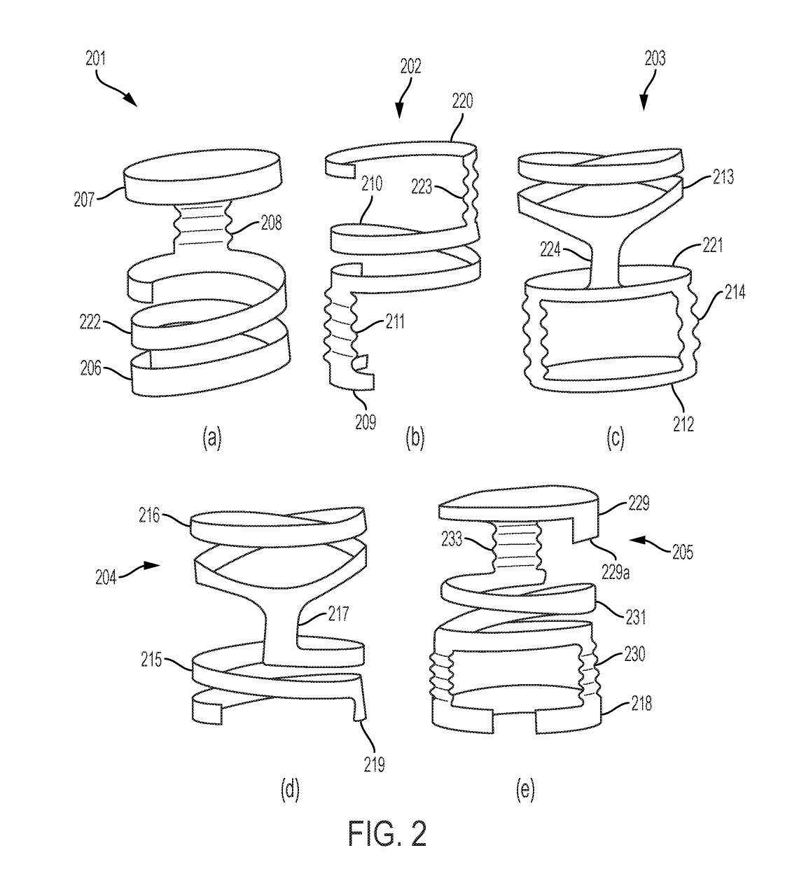

The brace may be configured in different ways, with alternative pelvic members, thoracic members and compliance connector configurations being used to achieve different design objectives as described in detail Appendix B, Chapter 7. For example, referring to FIG. 2, a number of alternative designs are shown. (It should be noted that brace 205 in FIG. 2(e) is the same as brace 100 in FIG. 1.) As mentioned above, the compliant connector(s) may be discrete or they may be integrated with the pelvic and thoracic members. For simplicity, the illustrations in FIGS. 1 and 2 show essentially homogenous material used for the pelvis/thoracic members and the complaint connectors.

In one embodiment, the pelvic members may wrap around entirely around the body or just a portion of the body. For example, referring to FIG. 2(e) brace 205, the pelvic member 218 is configured to wrap around most of the body, but remains open in the front as shown. Likewise, FIG. 2(d) brace 204 has a pelvic member 219 that wraps around the user's hip but is open in the front. Such an embodiment may also require additional apparatus, such as a belt or strap, to secure the pelvic member to the user. Alternatively, the pelvic member may fully wrap around the user. For example, FIG. 2(c) brace 203 has a circular pelvic member 212 which wraps around the user. Similarly, FIG. 2(a) brace 201 has a helical pelvic member 206 which wraps entirely around the user.

FIG. 2(b) brace 202 shows another embodiment in which the pelvic member does not wrap around the patient, but rather is configured as a pad 209 to apply pressure to a particular point on the user's hip/pelvis. Such an embodiment may also require additional apparatus, such as a belt or strap, to secure the pelvic member to the user.

Like the pelvic member, the thoracic member may be configured in different ways. In one embodiment the thoracic member wraps around the user's body. For example, braces 201 and 205 have circular thoracic members 207, 229, which wrap around the user's upper chest. Brace 205 also has an additional pad 229a extending from the thoracic member 229 for additional contact surface to spread the load from the compliant connectors as discussed below. Such embodiments may be preferred to provide specified force to particular areas of the spine. Likewise, braces 203 and 204 have crossed helix thoracic members 213, 216. Such embodiments may be preferred to provide larger ranges of motion in sagittal bending. Alternatively, the thoracic member may be open as with brace 202. Thoracic member 220 of brace 202 just partially wraps around the user's upper chest. Such embodiment may be preferred for Scoliotic curves with an apex opposite member 223. Such an embodiment may also require additional apparatus, such as a belt or strap, to secure the thoracic member to the user.

A variety of different compliant connector configurations are possible to connect and bias the thoracic member and pelvic member. The braces in FIG. 2 use a variety of different compliant connectors, including helix members, cross helix members, single curve members, and single/double corrugated members. For example, braces 201, 202, 204 and 205 each use a helix member 222, 210, 215, 231, respectively, as one of several compliant connectors. Helixes may be used to impart a torsional constraint force between the pelvic member and thoracic member, while allowing motion in the sagittal and lateral directions. In braces 201 and 206, the helix member 222, 215 is integrated with the pelvic member 206, 219, respectively, while braces 202 and 205 have a discrete helix member 210, 231 positioned proximate the lower thorax of the user. Braces 201, 202, 203, and 205 also use single/double corrugated members 208, 231/223, 214, 230/233 as compliant connectors. Single/double corrugated members may be used to impart lateral bending force between the pelvic member and thoracic member. Braces 201 and 205 both use single/double corrugated members 208, 233 in the back of the brace to connect the thoracic member to an intermediate compliant connector, in the case helix members 222, 231. Likewise, brace 202 uses single/double corrugated member 223 to connect thoracic member 220 to helix member 210. Using single/double corrugated members to connect to the thoracic member may be preferred to permit twisting while applying sagittal bending forces

Single/double corrugated members may also be used to connect to the pelvic member. For example, braces 203, 205 use two single/double corrugated members 214, 230 to connect to the pelvic member 212, 218. Using symmetrical single/double corrugated members may be preferred for permit twisting and in-plane bending while providing lateral bending force. Likewise, brace 202 uses one single/double corrugated member 211 to connect to the pelvic member 209. Using single/double corrugated members to connect to the pelvic member may be preferred to permit sagittal bending while providing limited force in the lateral bending direction.

In yet other embodiments, braces 203, 204 use a curved member 224, 217 as intermediate compliant connectors. Using curved members as intermediate compliant connectors may be preferred for permitting sagittal bending while applying lateral bending and twisting force.

The various thoracic members, pelvic members, and compliant connectors described in connection with FIGS. 2(a)-(e) may be mixed and matched to form different embodiments. Still other embodiments involving mixing and matching these different components will be obvious of those of skill in the art in light of this disclosure.

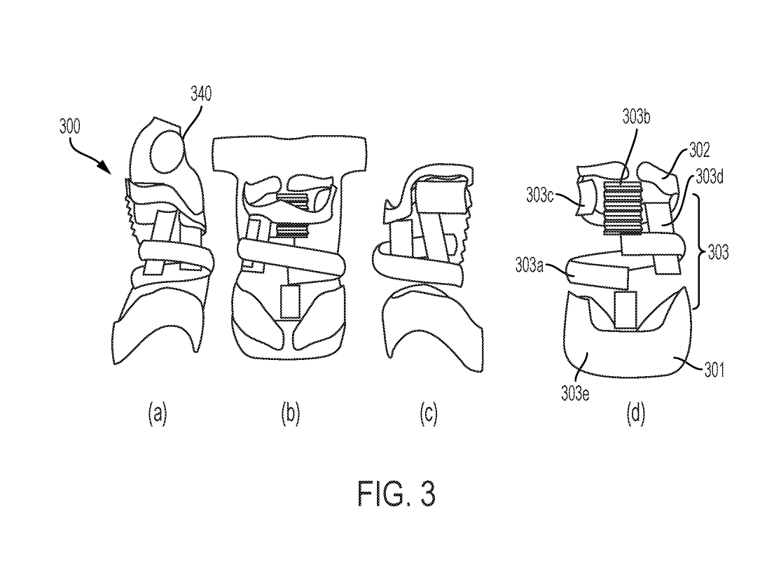

FIGS. 3(a)-(d) show one embodiment of the brace 300 of the present invention superimposed on a human torso 340. FIG. 3(a) shows the brace 300 on the torso 340 from the right side view. FIG. 3(b) shows the brace 300 on the torso 340 from the front, FIG. 3(c) shows the brace from the left side without being superimposed on the body and FIG. 3(d) shows the brace from the rear without being superimposed on the torso. This embodiment, like the embodiments described above, comprises a pelvic 301 and a thoracic 302 with compliant connectors 303 connecting the pelvic member 301 to the thoracic member 302 and providing a resistive force between them. In this particular embodiment, the compliant connectors comprise a helical member 303(a), a corrugated member 303(b), a curved member 303(c), a curved member 303(d), and a cantilevered member 303(e). It should be understood, that the type of compliant connector 303, its configuration, and its placement relative to other complaint connectors and the pelvic and thoracic members provides the characteristic corrective forces of a particular brace. Those of skill in the art will understand that modifying the compliant connector type, its material and its position within the brace, will affect the corrective forces.

In one particular embodiment of the brace 300, helix 303a was constructed with 12 layers of carbon fiber a layer thickness of 0.305 mm. The carbon was laid directly on top of the mold and the entire mold was vacuum bagged. This 12 layer helix had thickness varying from 3.8 to 4.1 mm. The force generators 303c and 303d were all produced using vacuum forming using PLA. The thickness varied between 1.8 mm to 3.0 mm for 303c and 303d.

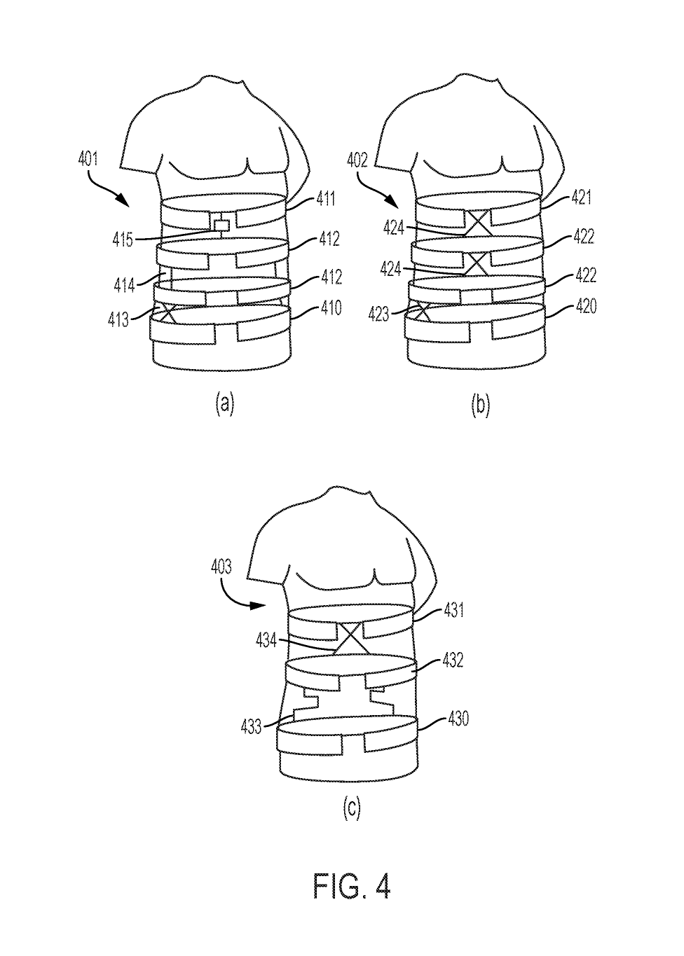

FIG. 4 shows an alternative embodiment of the brace of the present invention which uses flexure connections. Using flexure mechanisms as complaint connectors provides for corrective forces as described for example in Appendix A generally and Appendix B, Chapter 4. FIGS. 4(a) through (c) show braces 401, 402 and 403, respectively, each using flexure mechanism for corrective forces. Specifically, referring to FIG. 4(a), the brace 401 comprises a pelvic member 410 and a thoracic member 411, and intermediate members 412. Various fixture mechanisms interconnect the various members. Specifically, a pair of cartwheel hinge complaint connectors 413 connects the pelvic member 410 with intermediate member 412. Intermediate members 412 are connected by a pair of single beam compliant connectors 414. And finally, the thoracic member 411 is connected with intermediate member 412 using a CT joint complaint connector 415.

Referring to FIG. 4B, brace 402 is shown having essentially the same pelvic thoracic and intermediate members 420, 421, 422, but having different compliant connectors. Specifically, like brace 401, the pelvic member 420 is connected to intermediate member 422 using a pair of cartwheel hinge complaint connectors 423. The intermediate members 422 are interconnected by a single cartwheel hinge complaint connector 424. Likewise, the thoracic member 421 and the intermediate member 422 is also connected by a single cartwheel hinge complaint connector 424. Referring to FIG. 4C, brace 403 comprises a pelvic member 430 and thoracic member 431 and an intermediate member 432. The pelvic member 430 and the intermediate member 432 are connected by a pair of cross pivot hinge complaint connectors 433. The thoracic member 431 is connected to intermediate member 432 using single cartwheel hinge complaint connector 434.

The various thoracic members, pelvic members, and flexure compliant connectors described in connection with FIG. 4, or anywhere else in this disclosure including the appendences, may be mixed and matched to form different embodiments. Still other embodiments involving mixing and matching these different components will be obvious of those of skill in the art in light of this disclosure.

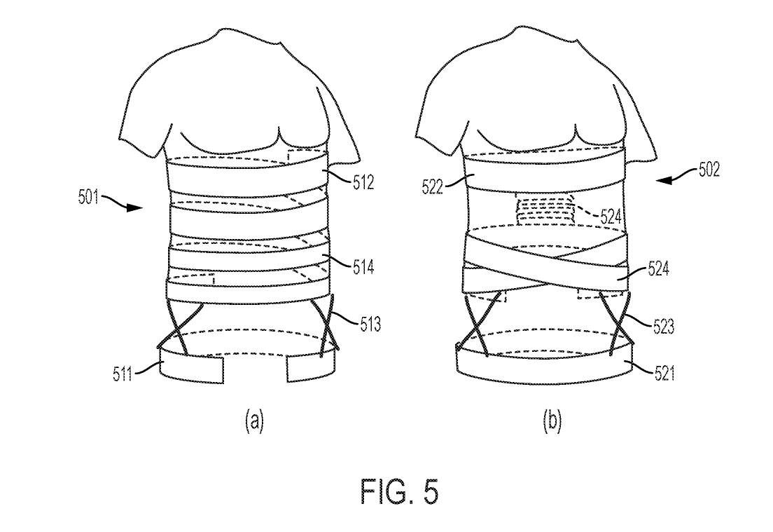

Referring to FIGS. 5(a) and 5(b), alternative embodiments of the brace of the present invention is shown in which a combination of flexure and shell compliant connectors is used. Specifically, referring to FIG. 5(a), brace 501 comprises a pelvic member 511 and a thoracic member 512 with a combination of flexure and shell compliant members connecting the two together. Specifically, a pair of cartwheel hinges 513 is connected to the pelvic member. A helical strip is connected to the cartwheel hinges 513 and is integral with the thoracic member 512. Referring to FIG. 5(b), brace 502 is shown in which the pelvic member 521 is connected to a pair of cartwheel hinges 523, which, in turn, is connected to a crossed helix 524. The crossed helix 524 is connected to the thoracic member 522 by corrugated member 524. It should be understood that, in addition to these two embodiments, the various flexure and shell mechanisms disclosed in the patent application and attached appendences can be mixed and matched to form of variety of different brace configurations having different corrective forces which can be tailored for a particular patient.

It should be understood that the foregoing is illustrative and not limiting and that obvious modifications may be made by those skilled in the art without departing from the spirit of the invention. Accordingly, the specification is intended to cover such alternatives, modifications, and equivalence as may be included within the spirit and scope of the invention as defined in the following claims.

What is claimed is:

1. A back brace comprising:
a pelvic member configured to be secured to a user such that said pelvic member is essentially immobilized relative to the pelvis of said user;
a thoracic member configured to around the user such that said thoracic member is essentially immobilized relative to the thorax of said user; and
one or more compliant connectors between said pelvic member and said thoracic member and configured to provide a resistive force between said pelvic member and said at least one thoracic member.

2. The back brace of claim 1, wherein said compliant connectors comprise at least one of a shell mechanism or a flexure mechanism.

3. The back brace of claim 2, wherein said compliant connectors comprise at least one shell mechanism from the group cross helix member, helical strip, single curve member, hyperbolic paraboloid member, double paraboloid member, single corrugated member or double corrugated member.

4. The back brace of claim 2, wherein said compliant connectors comprise at least one fixture mechanism from the group cartwheel hinge, parallel beam, cross pivot hinge, cross beam, LET outside, LET inside, and S-beam.

5. The back brace of claim 1, wherein said pelvic member and said at least one thoracic member are integrally formed with said one or more first compliant connectors.

6. The back brace of claim 1, wherein said one or more first compliant connectors is discrete from said pelvic member and said thoracic member.

7. The back brace of claim 6, wherein said pelvic member is rigid and said thoracic member are rigid.

8. The back brace of claim 1, wherein said pelvic member and said thoracic member wrap completely around said user.

9. The back brace of claim 1, wherein said one or more first complaint connectors facilitates movement of said pelvic member to said thoracic member about a rotational axis.

10. The back brace of claim 9, wherein said rotational movement is at least about 10 degrees.

11. The back brace of claim 10, wherein said rotational movement is at least about 11 degrees.

12. The back brace of claim 9, wherein said first complaint connectors comprise a revolute joint to facilitate movement about said rotational axis.

13. The back brace of claim 12, wherein each of said complaint connectors comprises at least a cartwheel hinge to facilitate said revolute joint.

14. The back brace of claim 9, wherein said first complaint connectors comprise a translational joint to facilitate translation movement of said rotational axis.

15. The back brace of claim 9, wherein said first complaint connectors comprise at least one compliant transitional joint to facilitate translation motion of said rotational axis.

16. The back brace of claim 1, wherein said first complaint connectors are configured to provides a revolute degree of freedom about a rotational axis and translation degree of freedom of said rotational axis.

17. A back brace comprising:
a pelvic member configured to contour a user such that said pelvic member is essentially immobilized relative to the pelvis of said user;
a thoracic member configured to contour the user such that said at least one thoracic member is essentially immobilized relative to said ribs of said user; and
at least one compliant connector between said pelvic member and said thoracic member, and being configured to allow said pelvic member and said thoracic member to move relative to each other with at least 2 degrees of freedom, but less than 6 degrees of freedom.

18. The back brace of claim 17, wherein said at least one compliant connector is configured to allow said pelvic member and said thoracic member to move relative to each other with at least 2 degrees of freedom, but less than 5 degrees of freedom.

19. The back brace of claim 18, wherein said at least one compliant connector is configured to allow said pelvic member and said thoracic member to move relative to each other with at least 2 degrees of freedom, but less than 4 degrees of freedom.

20. The back brace of claim 17, wherein said at least one compliant connector is configured to provide at least a 30N resistive force between said pelvic member and said thoracic member.
